(19) United States
(12) Patent Application Publication
(10) Pub. No.: US 2015/0004249 A1
(43) Pub. Date: Jan. 1, 2015

(54) INSULIN-MIMETIC LOCAL THERAPEUTIC ADJUNCTS FOR ENHANCING SPINAL FUSION

(71) Applicant: Rutgers, The State University of New Jersey, New Brunswick, NJ (US)

(72) Inventors: Sheldon S. Lin, Chatham, NJ (US); John Koerner, Newark, NJ (US); Michael J. Vives, Newark, NJ (US); Joseph Benevenia, Montclair, NJ (US); Eric Breitbart, South Orange, NJ (US)

(21) Appl. No.: 14/489,642

(22) Filed: Sep. 18, 2014

Related U.S. Application Data
(63) Continuation of application No. PCT/US13/66895, filed on Oct. 25, 2013.
(60) Provisional application No. 61/718,646, filed on Oct. 25, 2012.

(51) Int. Cl. A61K 33/30; A61K 47/46; A61K 33/24

(52) U.S. Cl. CPC A61K 33/30; A61K 33/24; A61K 47/46
USPC 424/641; 424/646; 423/555; 623/17.16

(57) ABSTRACT

Bone tissue materials comprising insulin-mimetic agents, such as suitable zinc, vanadium, tungsten, molybdenum, niobium, selenium, and manganese compounds, for facilitating spinal fusion of vertebrae in spinal fusion surgical procedures, and methods thereof. Additionally provided is a bone tissue kit for facilitating fusion of vertebrae in a spinal fusion surgical procedure including a composition formulated for facile application in a spinal fusion procedure comprising an insulin-mimetic agent and a pharmaceutically acceptable carrier. Yet further provided is an implantable device for enhancing spinal fusion including a prosthetic implant configured to stabilize and promote the fusion of two adjacent vertebrae, wherein the bone tissue contacting surfaces of the prosthetic implant are coated with a composition comprising an insulin-mimetic agent.

### FIELD OF THE INVENTION

The present invention relates to use of insulin-mimetic agents as therapeutic adjuncts for enhancing spinal fusion, bone tissue materials and methods used for enhancing spinal fusion in surgical procedures.

### BACKGROUND OF THE INVENTION

Spinal fusion is a common procedure performed for a variety of conditions including spondylosis, disk disorders, and spinal stenosis. The rates of pseudoarthrosis after single level spinal fusion have been reported up to 35%. The process of osteogenesis after spinal arthrodesis is similar to that which occurs during fracture healing and heterotopic ossification, and agents that increase the rate of fusion have an important role in decreasing pseudoarthrosis following spinal fusions. Previous studies found that insulin or insulin-like growth factor treatment can stimulate fracture healing in diabetic and normal animal models. Small molecule therapies that can mimic the effects of insulin or insulin-like growth factor could produce the same beneficial effects on bone regeneration.

Several studies have validated a posterolateral intertransverse lumbar spinal fusion model in the rat. This model has been used to study effects of bone morphogenetic proteins on spinal fusion, and has been used more recently to assess the effects of pharmacologic agents on fusion healing. The benefits of this model include low cost and good reproducibility.

Studies by Dedania et al. analyzed the effects of a time released local insulin implant in a rat segmental defect model. (Dedania J, et al., J. Orthop. Res. 2011, 29:92-99.) Defects treated with a time released insulin implant had significantly more new bone formation and greater quality of bone than those treated with palmitic acid alone seen on histology and histomorphometry. The local microenvironment and growth factor levels are critical for any osseous fusion. For example, studies by Vermnna et al. analyzed the levels of growth factors in the fusion site of diabetics undergoing hindfoot fusion. (Verma R, et al., Current Orthopaedic Practice 2011, 22: 251-256.) Samples were taken at the time of fusion, and patients were followed clinically for signs of fusion. They observed decreased levels of growth factors, specifically PDGF-AB and VEGF, in patients that went on to non-union. To our knowledge, prior to this invention, no in vivo evaluation of therapy on spinal fusion by local administration of an insulin-mimetic agents, such as zinc or vanadium, has been performed.

### SUMMARY OF THE INVENTION

The present invention provides a unique strategy to facilitate spinal fusion in spinal fusion procedures. In one aspect the present invention provides a bone tissue material for facilitating fusion of vertebrae in a spinal fusion surgical procedure, the material containing an insulin-mimetic agent. In one embodiment, the bone tissue material contains autograft bone tissue. In another embodiment, the bone tissue material contains allograft bone tissue.

In another aspect the present invention provides a surgical procedure for stabilizing vertebrae in a spine, including the steps of:
exposing a portion of each of adjacent vertebrae; and
placing supplementary bone tissue material and an insulin-mimetic agent within an area between the exposed portions of the adjacent vertebrae and in contact with the exposed portions of both vertebrae;
wherein the insulin-mimetic agent is provided in an amount effective to increase the rate of fusion of the two vertebrae with the bone tissue material.

In one embodiment, the vertebrae are lumbar vertebrae. In another embodiment, the vertebrae are cervical vertebrae. In one embodiment, the bone tissue material contains autograft bone tissue. In another embodiment, the bone tissue material contains allograft bone tissue. In one embodiment, the insulin-mimetic agent is mixed with the bone tissue material. In a specific embodiment, the bone tissue material is autograft bone tissue and the insulin-mimetic agent is mixed with the bone tissue material after harvesting and before being placed between the exposed portions of the two vertebrae.

In another embodiment, the method further includes the step of supporting the two vertebrae with a prosthetic implant configured to stabilize the two vertebrae and promote fusion of the two vertebrae with the bone tissue material. In one embodiment, the bone tissue contacting surfaces of the prosthetic implant are coated with the insulin-mimetic agent.

In another aspect the present invention provides a bone tissue kit for increasing the rate of fusion of vertebrae in a spinal fusion surgical procedure, including the composition containing an insulin-mimetic agent and a pharmaceutically acceptable carrier. In an embodiment the kit also contains allograft bone tissue material. In one embodiment the insulin-mimetic agent and the allograft bone tissue material are provided in a mixture. In another embodiment, the insulin-mimetic agent and allograft bone tissue material are provided for subsequent mixing. In another aspect the present invention provides a composition for increasing the rate of spinal fusion in a spinal fusion surgical procedure, wherein the composition contains an insulin-mimetic agent and a pharmaceutically acceptable carrier. In one embodiment, the composition contains allograft bone material

In another aspect the present invention provides an implantable device for enhancing spinal fusion, in which a prosthetic implant is configured to stabilize and promote the fusion of two adjacent vertebrae, wherein the bone tissue contacting surfaces of the prosthetic implant are coated with a composition comprising an insulin-mimetic agent.

Examples of insulin mimetic agents suitable for the present invention include, but are not limited to, suitable zinc, vanadium, tungsten, molybdenum, niobium, selenium, or manganese compounds.

The present invention thus provides a unique method for enhancing spinal fusion in a patient, preferably mammalian animal and more preferably a human, either diabetic or non-diabetic. Development of an insulin-mimetic therapy of the present invention would obviate the need for developing specialized methods to deliver complex molecules, such as growth factors like insulin, and thereby reduce costs, eliminate specialized storage, and enhance ease of use. These and other aspects of the present invention will be better appreciated by reference to the following drawings and detailed description.

### BRIEF DESCRIPTION OF THE DRAWINGS

FIG. 1 illustrates the transverse processes of L4-L5 were cleaned of soft tissue, and decorticated with a high-speed burr

FIG. 2 illustrates the crushed autograft was then spread over and between the transverse processes at the appropriate level (L4-L5). An equivalent amount of implant, or blank was incorporated into the autograft bed

FIG. 3 illustrates radiographs of the vanadium-treated spines in the rat model in comparison with those in the control group.

FIG. 4 is a graph showing the radiographic test results.

FIG. 5 is a graph showing the manual palpitation test results.

### DETAILED DESCRIPTION OF THE INVENTION

In exploiting the biological impact of insulin-mimetic agents on bone, we found that these agents play a critical role in bone healing. The present invention thus uses an insulin-mimetic agent, such as a vanadium or zinc compound, to enhance spinal fusion, for example in treating spinal arthrodesis. The insulin-mimetic agents suitable for the present invention include, but are not limited to, zinc, vanadium, tungsten, molybdenum, niobium, selenium, or manganese metal or compounds.

Thus, in one aspect, the present invention provides a bone tissue material, ceramic bone-graft substitute, or mixture thereof for facilitating fusion of vertebrae in a spinal fusion surgical procedure containing an insulin-mimetic agent. Bone tissue material suitable for use in the present invention includes both autograft and allograft materials.

In one embodiment of this aspect, the bone tissue material contains an insulin-mimetic agent selected from zinc, vanadium, tungsten, molybdenum, niobium, selenium, and manganese compounds.

In another embodiment of this aspect, the bone tissue material contains an insulin-mimetic agent selected from vanadium and zinc compounds.

In another embodiment of this aspect, the bone tissue material further contains a pharmaceutically acceptable carrier.

In another embodiment of this aspect, the pharmaceutically acceptable carrier is an inorganic salt.

In another embodiment of this aspect, the pharmaceutically acceptable carrier is an inorganic salt selected from sulfates and phosphates.

In another embodiment of this aspect, the pharmaceutically acceptable carrier is a calcium salt.

In another aspect, the present invention provides a spinal fusion procedure utilizing an insulin mimetic agent for enhancing spinal fusion. In one embodiment, a surgical procedure for stabilizing vertebrae in a spine is provided, including the steps of exposing a portion of each of adjacent vertebrae; and placing supplementary bone tissue material, ceramic bone-graft substitute, or mixture thereof and an insulin-mimetic agent within an area between the exposed portions of the adjacent vertebrae and in contact with the exposed portions of both vertebrae; wherein the insulin-mimetic agent is provided in an amount effective to increase the rate of fusion of the two vertebrae with the bone tissue material.

In one embodiment of this aspect, the insulin-mimetic agent is a zinc, vanadium, tungsten, molybdenum, niobium, selenium, or manganese compound.

In another embodiment of this aspect, the insulin-mimetic agent is a zinc or vanadium compound.

In another embodiment of this aspect, the insulin-mimetic agent is added to the supplementary bone tissue material and/or ceramic bone-graft substitute to provide a supplementary bone tissue material containing the insulin-mimetic agent.

In another embodiment of this aspect, the insulin-mimetic agent is added separately from the supplementary bone tissue material and/or ceramic bone-graft substitute as a composition further comprising a pharmaceutically acceptable carrier. According to one embodiment, the composition is an insulin-mimetic calcium sulfate pellet.

In another embodiment of this aspect, the method is in combination with transplantation of an autograft bone, allograft bone or a ceramic bone-graft substitute. According to one embodiment, an insulin-mimetic agent is admixed with the autograft, allograft or a ceramic bone-graft substitute

In another embodiment of this aspect, the method is in combination with implantation of an interbody device. According to one embodiment, the interbody device is a prosthetic implant configured to stabilize two adjacent vertebrae and promote fusion of the two vertebrae. According to one embodiment, the interbody device can be used in combination with an autograft bone, allograft bone or a ceramic bone-graft substitute. According to one embodiment, an insulin-mimetic agent is admixed with the autograft, allograft or a ceramic bone-graft substitute. In another embodiment, the bone tissue contacting surfaces of the prosthetic implant are coated with the insulin-mimetic agent and may be used with or without the autograft bone, allograft bone or ceramic bone-graft substitute, which may or may not be admixed with an insulin-mimetic agent.

In another aspect, the present invention provides a bone tissue kit for facilitating fusion of vertebrae in a spinal fusion surgical procedure, including a composition containing an insulin-mimetic agent and a pharmaceutically acceptable carrier. In an embodiment the kit also contains allograft bone tissue material and/or ceramic bone-graft substitute. In one embodiment the insulin-mimetic agent and the allograft bone tissue material and/or ceramic bone-graft substitute are provided in a mixture. In another embodiment, the insulin-mimetic agent and allograft bone tissue material or ceramic bone-graft substitute are provided for subsequent mixing.

In one embodiment of this aspect, the insulin-mimetic agent is selected from zinc, vanadium, tungsten, molybdenumm, niobium, selenium, and manganese compounds, and combinations thereof. The insulin-mimetic agent can be in any form known in the art that is suitable for use in spinal fusion procedures.

In another aspect, the present invention provides a composition comprising an insulin-mimetic agent for enhancing spinal fusion in a spinal fusion surgical procedure, wherein the composition contains an insulin-mimetic agent and a pharmaceutically acceptable carrier. In one embodiment, the composition contains allograft bone material and for ceramic bone-graft substitute.

In one embodiment of this aspect, the insulin-mimetic agent is selected from zinc, vanadium, tungsten, molybdenum, niobium, selenium, and manganese compounds, and combinations thereof.

In another aspect, the present invention provides an implantable device for enhancing spinal fusion, in which a prosthetic implant is configured to stabilize and promote the fusion of two adjacent vertebrae, wherein the bone tissue contacting surfaces of the prosthetic implant are the device coated with a composition comprising an insulin-mimetic agent. The device may also be configured to supply autograft bone, allograft bone or ceramic bone-graft substitute to the exposed surfaces of the two adjacent vertebra, which bone or bone-graft substitute may or may not be admixed with an insulin-mimetic agent

In one embodiment of this aspect, the insulin-mimetic agent is selected from zinc, vanadium, tungsten, molybdenum, niobium, selenium, or manganese compounds, and combinations thereof.

Examples of diseases or conditions that make a patient in need of spinal fusion include, but are not limited to, arthrodesis, degenerative disc disease, spinal disc herniation, discogenic pain, spinal tumor, vertebral fracture, scoliosis, kyphosis (i.e., Scheuermann's disease), spondylolisthesis, spondylosis, Posterior Rami Syndrome, other degenerative spinal conditions, and any other conditions that cause instability of the spine.

Optionally, the treatment method of the present invention is combined with at least one procedure selected from bone autograft, bone allograft, autologous stem cell treatment, allogeneic stem cell treatment, chemical stimulation, electrical stimulation, internal fixation, and external fixation in order to stabilize the fused vertebrae or increase the rate at which the two adjacent vertebrae fuse together.

The insulin-mimetic zinc compounds suitable for the present invention include inorganic zinc compounds, such as mineral acid zinc salts. Examples of inorganic zinc compounds include, but are not limited to, zinc chloride, zinc sulfate, zinc phosphate, zinc carbonate, and zinc nitrate, or combinations thereof.

The insulin-mimetic zinc compounds can also be zinc salts of organic acids. Examples of organic acid zinc salts include, but are not limited to, zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6 mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)2], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ), or the like, or combinations thereof. In another embodiment, the organic acid of zinc salt is a naturally occurring fatty acid.

Suitable organovanadium-based insulin-mimetic agents include, but are not limited to, vanadyl acetylacetonate (VAC), vanadyl sulfate (VS), vanadyl 3-ethyl-acetylacetonate (VET), and bis(maltolato)oxovanadium (BMOV), and the like. In a preferred embodiment, the organovanadium compound is vanadyl acetylacetonate (VAC). Vanadyl acetylacetonate (VAC), an organic vanadium compound, has demon-strated insulin-mimetic effects in type 1 and type 2 diabetic animals and human studies and prevented some of the associated complications of diabetes in animal studies. Additional pharmacological activities of VAC, which have been studied, include the inhibition of gluconeogenesis, a decrease in glutamate dehydrogenase activity, and antilipolysis. Use of these vanadium-based insulin-mimetic agents to accelerate bone healing or regeneration, or as therapeutic adjuncts for cartilage injury and repair, has been previously disclosed by the present inventors in U.S. Provisional Application Nos. 61/295,234 and 61/504,777; and PCT Application Nos. PCT/US 11/21296 and PCT/US12/45771, which are hereby incorporated by reference in their entirety.

Suitable tungsten, selenium, molybdenum, niobium, or manganese compounds as insulin mimetics for bone healing or regeneration are also encompassed by the present disclosure, and their forms and administration modes are within the grasp of an ordinary skill in the art.

Examples of tungsten compounds include, but are not limited to, sodium tungstate [Na₂WO₄.xH₂O], tungstophosphoric acid [H₃[P(W₃O₁₀)₄].xH₂O], alanine complex of tungstophosphoric acid (WPA-A) [H₃[P(W₃O₁₀)₄][(CH₃CH(NH₂)COOH].xH₂O], homo-polyoxotungstates and vanadium polyoxotungstates, tungsten (VI) perooxo complexes (e.g., (gu)₂[WO₂(O₂)₂] and (gu)[WO(O₂)₂(quin-2-c)], wherein “gu” is guanidinium and “quin-2-c” is quinoline 2-carboylate), and permetalloxide of tungstate (pW). Molybdenum compounds include, for example, permetalloxide of molybdate.

Niobium compounds include, but are not limited to, Nb(V) peroxo complexes, e.g., (gu)₃[Nb(O₂)₄] and (gu)₂[Nb(O₂)₃(quin-2-c), wherein “gu” is guanidinium and “quin-2-c” is quinoline 2-carboylate.

Selenium compounds include, but are not limited to, sodium selenate [NazSeO₄.xH2O] and sodium selenite [Na₂SeO₃.xH₂O].

Manganese compounds include, but are not limited to, 3-O-methyl-D-chiro-inositol+manganese chloride (MnCl₂), D-chiro-inositol+manganese chloride (MnCl₂), manganese sulfate [MnSO4], inositol glycan pseudo-disaccharide Mn(2+) chelate containing D-chiro-inositol 2a (as pinitol) and galactosamine, oral manganese, manganese oxides, e.g., MnO₂, MnOAl₂O₃, and Mn₃O₄.

It will be appreciated that actual preferred amounts of a pharmaceutical composition used in a given therapy will vary depending upon the particular form being utilized, the particular compositions formulated, the mode of application, and the particular site of administration, and other such factors that are recognized by those skilled in the art including the attendant physician or veterinarian. Optimal administration rates for a given protocol of administration can be readily determined by those skilled in the art using conventional dosage determination tests.

Dosages of an insulin-mimetic suitable for the present invention may vary depending on the particular use envisioned. The determination of the appropriate dosage or route of administration is well within the skill of an ordinary physician.

The route of administration of “local zinc” via “insulin mimetic delivery system” is in accordance with known methods, e.g. via immediate-release, controlled-release, sustained-release, and extended-release means. Preferred modes of administration for the insulin-mimetic delivery system include injection directly into a fusion site and areas adjacent and/or contiguous to these sites, or surgical implantation of insulin-mimetic agent(s) directly into the fusion sites and area adjacent and/or contiguous to these sites. This type of system will allow temporal control of release as well as location of release as stated above.

The formulations used herein may also contain more than one active compound as necessary for the particular indication being treated, preferably those with complement-ary activities that do not adversely affect each other. Alternatively, or in addition, the formulation may comprise a cytotoxic agent, cytokine or growth inhibitory agent. Such molecules are present in combinations and amounts that are effective for the intended purpose.

Vanadium, which exists in +4 (vanadyl) and +5 (vanadate) compounds in the biological body, have demonstrated poor absorption rates within the gastrointestinal (GI) tract and GI side-effects, such as diarrhea and vomiting. As a result, additional organic vanadium compounds, i.e., vanadyl 3-ethylacetylacetonate (VET), bis(maltolato)oxo-vanadium (BMOV), and VAC, have been synthesized in order to improve absorption and safety. VAC with an organic ligand has been proven to be more effective in its anti-diabetic function compared with other vanadium compounds, including BMOV, VS, and VET.

Therapeutic formulations of vanadium compounds in the vanadium delivery systems employable in the methods of the present invention are prepared for storage by mixing the vanadium compound having the desired degree of purity with optional pharmaceutically acceptable carriers, excipients, or stabilizers (Remington's Pharmaceutical Sciences 16th edition, Osol, A. Ed. (1980)). Such therapeutic formulations can be in the form of lyophilized formulations or aqueous solutions. Acceptable biocompatible carriers, excipients, or stabilizers are nontoxic to recipients at the dosages and concentrations employed, and may include buffers, for example, phosphate, citrate, and other organic acids; antioxidants including ascorbic acid and methionine; preservatives (e.g. octadecyldimethylbenzyl ammonium chloride; hexa-methonium chloride; benzalkonium chloride, benzethonium chloride; phenol, butyl or benzyl alcohol; alkyl parabens, for example, methyl or propyl paraben; catechol; resorcinol; cyclohexanol; 3-pentanol; and m-cresol); low molecular weight (less than about 10 residues) polypeptides; proteins, for example, serum albumin, gelatin, or immunoglobulins; hydrophilic polymers, for example, polyvinylpyrrolidone; amino acids, for example, glycine, glutamine, asparagine, histidine, arginine, or lysine; monosaccharides, disaccharides, and other carbohydrates including glucose, mannose, dextrins, or hyaluronan; chelating agents, for example, EDTA; sugars, for example, sucrose, mannitol, trehalose or sorbitol; salt-forming counter-ions, for example, sodium; metal complexes (e.g. Zn-protein complexes); and/or non-ionic surfactants, for example, TWEEN™, PLURONICS® or polyethylene glycol (PEG).

In order for the formulations to be used for in vivo administration, they must be sterile. The formulation may be readily rendered sterile by filtration through sterile filtration membranes, prior to or following lyophilization and reconstitution. The therapeutic formulations herein preferably are placed into a container having a sterile access port, for example, an intravenous solution bag or vial having a stopper pierceable by a hypodermic injection needle.

The vanadium may also be entrapped in microcapsules prepared, for example by coacervation techniques or by interfacial polymerization, for example, hydroxy-methyl-cellulose or gelatin-microcapsules and poly-(methylmethacrylate) microcapsules, respectively. Such preparations can be administered in colloidal drug delivery systems (for example, liposomes, albumin microspheres, microemulsions, nano-particles and nanocapsules) or in macroemulsions. Such techniques are disclosed in Remington's Pharmaceutical Sciences, 16th Edition (or newer), Osol A. Ed. (1980).

Optionally, the organovanadium agent in the vanadium delivery systems includes a porous calcium phosphate, non-porous calcium phosphate, hydroxy-apatite, tricalcium phosphate, tetracalcium phosphate, calcium sulfate, calcium minerals obtained from natural bone, inorganic bone, organic bone, or a combination thereof.

Where sustained-release or extended-release administration of vanadium in the vanadium delivery systems is desired, microencapsulation is contemplated. Microencapsulation of recombinant proteins for sustained release has been successfully performed with human growth hormone (rhGH), interferon-α, -β, -γ (rhIFN-α, -β, -γ), interleukin-2, and MN rgp120. Johnson et al., Nat. Med. 2: 795-799 (1996); Yasuda, Biomed. Ther. 27: 1221-1223 (1993); Hora et al., Bio/Technology 8: 755-758 (1990); Cleland, “Design and Production of Single Immunization Vaccines Using Polylactide Polyglycolide Microsphere Systems” in Vaccine Design: The Subunit and Adjuvant Approach, Powell and Newman, eds., (Plenum Press: New York, 1995), pp. 439-462; WO 97/03692, WO 96/40072, WO 96/07399 and U.S. Pat. No. 5,654,010.

Suitable examples of sustained-release preparations include semipermeable matrices of solid hydrophobic polymers containing the vanadium in the vanadium delivery systems, which matrices are in the form of shaped articles, e.g. films, or microcapsules. Examples of sustained-release matrices include one or more polyanhydrides (e.g., U.S. Pat. Nos. 4,891,225; 4,767,628), polyesters, for example, polyglycolides, polylactides and polylactide-co-glycolides (e.g., U.S. Pat. No. 3,773,919; U.S. Pat. No. 4,767,628; U.S. Pat. No. 4,530,840; Kulkarni et al., Arch. Surg. 93: 839 (1966)), polyamino acids, for example, polylysine, polymers and copolymers of polyethylene oxide, polyethylene oxide acrylates, polyacrylates, ethylene-vinyl acetates, polyamides, polyurethanes, polyorthoesters, polyacetylnitriles, polyphosphazenes, and polyester hydrogels (for example, poly(2-hydroxyethyl-methacrylate), or poly(vinyl-alcohol)), cellulose, acyl substituted cellulose acetates, non-degradable polyurethanes, polystyrenes, polyvinyl chloride, polyvinyl fluoride, poly(vinylimidazole), chlorosulphonated polyolefins, polyethylene oxide, copolymers of L-glutamic acid and .gamma.-ethyl-L-glutamate, non-degradable ethylene-vinyl acetate, degradable lactic acid-glycolic acid copolymers, for example, the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release for over 100 days, certain hydrogels release proteins for shorter time periods. Additional non-biodegradable polymers which may be employed are polyethylene, polyvinyl pyrrolidone, ethylene vinylacetate, polyethylene glycol, cellulose acetate butyrate and cellulose acetate propionate.

Alternatively, sustained-release formulations may be composed of degradable biological materials, for example, collagen and derivatives thereof, bioerodible fatty acids (e.g., palimitic acid, steric acid, oleic acid, and the like). Biodegradable polymers are attractive drug formulations because of their biocompatibility, high responsibility for specific degradation, and ease of incorporating the active drug into the biological matrix. For example, hyaluronic acid (HA) may be crosslinked and used as a swellable polymeric delivery vehicle for biological materials. U.S. Pat. No. 4,957,744; Valle et al., Polym. Mater. Sci. Eng. 62: 731-735 (1991). HA polymer grafted with polyethylene glycol has also been prepared as an improved delivery matrix which reduced both undesired drug leakage and the denaturing associated with long term storage at physiological conditions. Kazuteru, M., J. Controlled Release 59:77-86 (1999). Additional biodegradable polymers which may be used are poly(caprolactone), polyanhydrides, polyamino acids, polyorthoesters, polycyanoacrylates, poly(phosphazines), poly(phosphodiesters), poly-esteramides, polydioxanones, polyacetals, polyketals, polycarbonates, polyortho-carbonates, degradable and nontoxic polyurethanes, polyhydroxylbutyrates, polyhydroxy-valerates, polyalkylene oxalates, polyalkylene succinates, poly(malic acid), chitin, and chitosan.

Alternatively, biodegradable hydrogels may be used as controlled-release materials for the vanadium compounds in the vanadium delivery systems. Through the appropriate choice of macromers, membranes can be produced with a range of permeability, pore sizes and degradation rates suitable for different types of vanadium compounds in the vanadium delivery systems.

Alternatively, sustained-release delivery systems for vanadium in the vanadium delivery systems can be composed of dispersions. Dispersions may further be classified as either suspensions or emulsions. In the context of delivery vehicles for a vanadium compound, suspensions are a mixture of very small solid particles which are dispersed (more or less uniformly) in a liquid medium. The solid particles of a suspension can range in size from a few nanometers to hundreds of microns, and include microspheres, microcapsules and nanospheres. Emulsions, on the other hand, are a mixture of two or more immiscible liquids held in suspension by small quantities of emulsifiers. Emulsifiers form an interfacial film between the immiscible liquids and are also known as surfactants or detergents. Emulsion formulations can be both oil in water (o/w) wherein water is in a continuous phase while the oil or fat is dispersed, as well as water in oil (w/o), wherein the oil is in a continuous phase while the water is dispersed. One example of a suitable sustained-release formulation is disclosed in WO 97/25563. Additionally, emulsions for use with a vanadium compound in the present invention include multiple emulsions, microemulsions, microdroplets and liposomes. Micro-droplets are unilamellar phospholipid vesicles that consist of a spherical lipid layer with an oil phase inside. E.g., U.S. Pat. No. 4,622,219 and U.S. Pat. No. 4,725,442. Liposomes are phospholipid vesicles prepared by mixing water-insoluble polar lipids with an aqueous solution.

Alternatively, the sustained-release formulations of vanadium in the vanadium delivery systems may be developed using poly-lactic-coglycolic acid (PLGA), a polymer exhibiting a strong degree of biocompatibility and a wide range of biodegradable properties. The degradation products of PLGA, lactic and glycolic acids, are cleared quickly from the human body. Moreover, the degradability of this polymer can be adjusted from months to years depending on its molecular weight and composition. For further information see Lewis, “Controlled Release of Bioactive Agents from Lactide/Glycolide polymer,” in Biogradable Polymers as Drug Delivery Systems M. Chasin and R. Langeer, editors (Marcel Dekker: New York, 1990), pp. 1-41.

The route of administration of “local vanadium” via a “delivery system” is in accordance with known methods, e.g. via immediate-release, controlled-release, sustained-release, and extended-release means. Preferred modes of administration for the organovanadium delivery system include injection directly into afflicted site and areas adjacent and/or contiguous to these site or surgical implantation of the organovanadium delivery system directly into afflicted sites and area adjacent and/or contiguous to these sites. This type of system may allow temporal control of release as well as location of release as stated above.

When an implantable device coated by a composite surface coating comprising an organovanadium compound is used, the coating can be formed by any methods known in the relevant art, for example, without limitation, those disclosed in Petrova, R. and Suwattananont, N., J. Electr. Mat., 34(5):8 (2005)). For example, suitable methods include chemical vapor deposition (CVD), physical vapor deposition (PVD), thermochemical treatment, oxidation, and plasma spraying (Fischer, R. C., Met. Progr. (1986); Habig, K. H., Tril. Int., 22:65 (1989)). A suitable coating of the present invention may also comprise combinations of multiple, preferably two or three, layers obtained by forming first boron diffusion coating followed by CVD (Z. Zakhariev, Z., et al., Surf. Coating Technol., 31:265 (1987)). Thermochemical treatment techniques have been well investigated and used widely in the industry. This is a method by which nonmetals or metals are penetrated by thermodiffusion followed by chemical reaction into the surface. By thermochemical treatment, the surface layer changes its composition, structure, and properties.

Other suitable coating techniques may include, but are not limited to, carburizing, nitriding, carbonitriding, chromizing, and aluminizing. Among these coating techniques, boronizing, being a thermochemical process, is used to produce hard and wear-resistant surfaces. As a person of ordinary skill in the art would understand, different coating techniques may be used to make the vanadium-based coatings and coated devices of the present invention in order to have desired properties suitable for specific purposes.

This study demonstrates the potential role of insulin-mimetics as bone graft enhancers using a rat posterolateral intertransverse lumbar fusion model. Significant differences between groups were demonstrated for radiographic analysis and manual palpation compared to controls. To our knowledge this is the first study of vanadium and zinc effects on spinal fusion.

Multiple studies have explored the effects of local insulin application on bone formation, for example, observation of more advanced healing microscopically in a rabbit fibula osteotomy model for animals injected with intravenous insulin, most notably in animals sacrificed between two and four weeks after surgery; observation of significant increase in bone formation indices in insulin-treated hemicalvariae after injecting insulin over the right hemicalvariae of adult mice for five days compared with the noninjected hemicalvariae; and observation that locally delivered Ultralente insulin increased callus mechanical strength in a non-diabetic rat femur fracture model. These studies, however, do not address the problem of systemic administration of insulin, or the short half-life of insulin when injected locally at the site of interest.

Because insulin without a carrier has a short half-life, the palmitic acid Linplant was proposed as a potential vehicle to deliver insulin at the appropriate site of action for an extended period of time. In an analysis of the effects of a time released local insulin implant in a rat segmental defect model, defects treated with the time released insulin implant had significantly more new bone formation and greater quality of bone than those treated with palmitic acid alone seen on histology and histomorphometry. In our studies we have confirmed the potential benefits of a time-released substance using a rat posterolateral transverse process fusion model. Significant differences were found with radiographs, manual palpation, and microCT in the insulin treatment groups versus controls. This study found similar results with insulin-mimetic agents of VAC and zinc.

The local environment is altered with the application of local insulin, as seen in our study. We found a significant increase in the ratio of IGF-I, an important growth factor in bone healing, to total protein at day four in the insulin treatment group. IGF-I has been studied previously as. Alteration of the local fracture environment (specifically increases of PDGF, TGF-BI, IGF-I and VEGF), has been seen with a local insulin depot system in the DM femur fracture model. Studies have demonstrated that IGF-I stimulates pre-osteoblastic cells, increases collagen expression while decreasing its degradation, and enhances fracture healing. When infused continuously into the arterial supply of a rat hind limb, a significant increase in cortical bone formation has been observed. In addition, it has been found that locally delivered IGF-I increased callus mechanical strength in a non-diabetic rat tibia fracture model. However, when infused into an estrogen replete rat model, continuous systemic infusion stimulated bone resorption rather than bone formation. These studies demonstrate the benefits of a local implant versus systemic administration.

Multiple studies have demonstrated the beneficial effects of osteoinductive growth factors such as rhBMP-2, rhBMP-7, and demineralized bone matrix on spinal fusions in an animal model. Our study also demonstrated increased rates of fusion based on the qualitative measures of radiographs and manual palpation with the addition of insulin-mimetics.

One potential issue using insulin-mimetics lies in its hypoglycemic action. Systemic blood glucose levels and hypoglycemia are concerning when applying an insulin-mimetic. However, previous studies have demonstrated that a time released insulin implant does not affect the systemic insulin, glucose, or glycosylated hemoglobin values. Our data supports this with little, if any, impact upon the systemic glucose values.

Osteoinductive growth factors, such as rhBMP-2 and rhBMP-7 add significant cost to surgical procedures, and concerns have been raised about the possibility of untoward effects. Insulin has also been studied extensively, and may serve as an inexpensive alternative to adjuncts of bone healing and fusion procedures. This study is the first to examine the effects insulin-mimetics in a rat spinal fusion model. Our results provide strong evidence that local insulin-mimetic agent delivery at the fusion site increases the rate of fusion and amount of bone formed in healthy normoglycemic rats.

Our study has demonstrated that local insulin-mimetics, such as vanadium and zinc compounds, enhance spinal fusion. Preliminary data has indicated that local insulin-mimetic treatment is an effective method to enhance spinal fusion in non-diabetic patients. Therefore, the invention can be used as a treatment regimen to increase fusion rates in patients undergoing spinal arthrodesis.

The present invention also finds wide application in veterinary medicines to enhance spinal fusion in a mammalian animal, including but not limited to, horses, dogs, cats, or any other domestic or wild mammalian animals. A particular useful application may be found, for example, in treating an injured race horse.

The following non-limiting examples illustrate certain aspects of the invention.

### EXAMPLES

Increased fusion rates were observed in a rat posterolateral lumbar spinal fusion model when treated with a time-released insulin implant in comparison with controls. The effects of insulin-mimetic agents were analyzed as an adjunct to spinal fusion in the rat posterolateral lumbar fusion model. Vanadyl acetylacetonate (VAC), or Zinc were made into a pellet with Calcium Sulfate, and applied to the fusion bed with autograft in a rat posterolataeral lumbar fusion. These were compared with a control group treated with autograft and a palmitic acid pellet.

### Materials and Methods

### Study Design

The protocol was approved by the animal institutional Care and Use Committee at UMDNJ-New Jersey Medical School. Fifty skeletally mature Sprague-Dawley rats weighing approximately 500 grams each underwent posterolateral intertransverse lumbar fusions with iliac crest autograft from L4-L5 utilizing a Wiltse-type approach. After exposure of the transverse processes and high-speed burr decortication, one of five pellets were added to the fusion site: a low dose Vanadium Calcium Sulfate pellet (0.75 mg/kg), a high dose Vanadium Calcium Sulfate pellet (1.5 mg/kg), a low dose Zinc Calcium Sulfate pellet (0.5 mg/kg), a high dose Zinc Calcium Sulfate pellet (1.0 mg/kg), and a control of micro-recrystalized palmitic acid pellet. An equal amount of iliac crest autograft (approximately 0.3 g per side) was harvested and implanted with each pellet. The rats were sacrificed at 8 weeks, and spines were harvested, removed of soft tissue, and tested by manual palpation, radiographs and MicroCT. All outcome parameters were independently reviewed by two separate individuals in a blinded manner and the lower grade of fusion was accepted when there was a discrepancy.

### Surgical Procedure

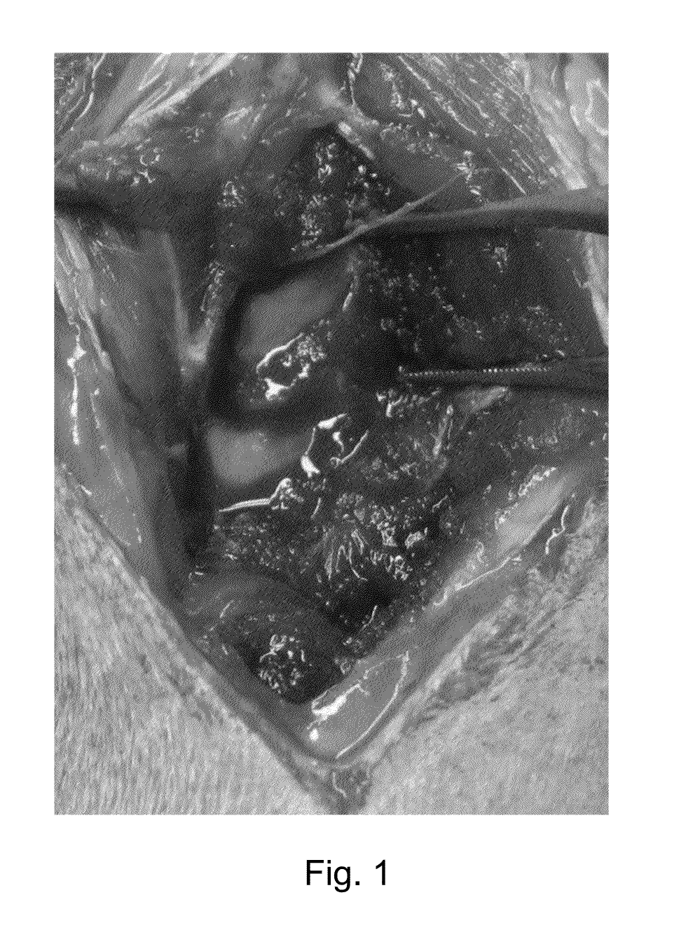

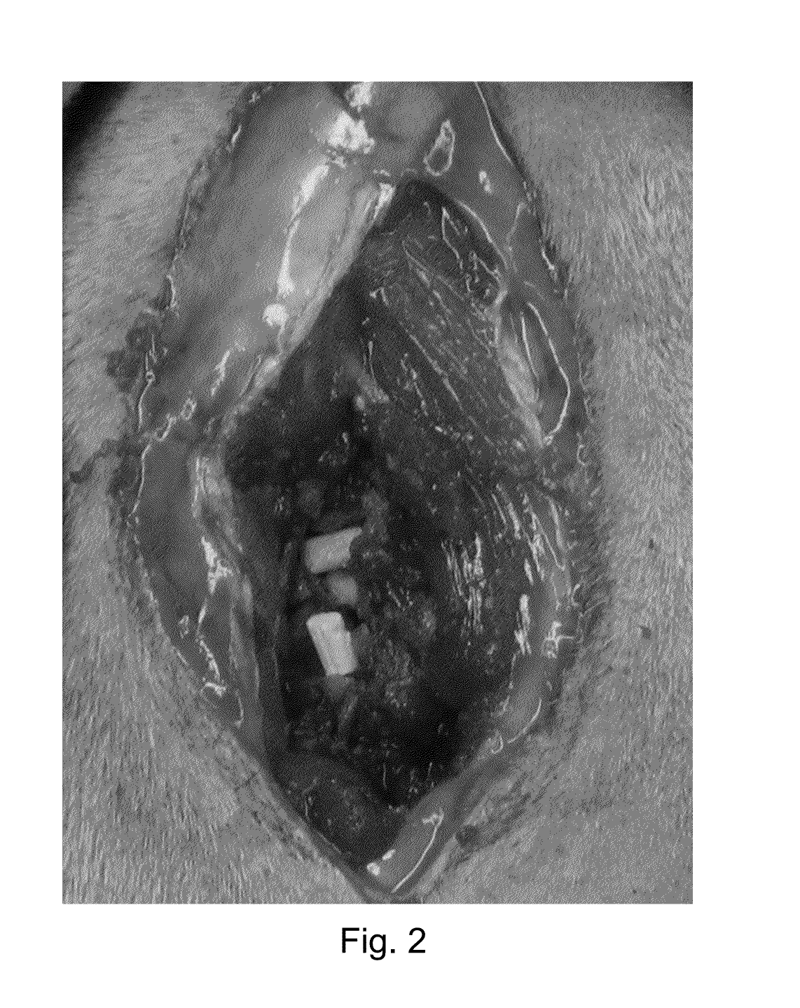

After obtaining general anaesthesia with intraperitoneal Ketamine (40 mg/kg) and Xylazine (5 mg/kg), the lumbar region of the rat was shaved and cleansed with povidone iodine soaked gauze. A dorsal midline incision was made from L3 to the sacrum. Two paramedian incisions were made through the lumbar fascia 5 mm from the midline. Dissection was taken to the iliac crest, and approximately 0.3 g of bone was harvested with small rongeurs. The harvested autograft was measured on a sterile scale in order to obtain 0.3 g per side. Blunt dissection was carried down posterolaterally, reflecting the paraspinal muscles lateral to the facet joints on each side. The reflected paraspinal muscles were held in place with retractors. The transverse processes of L4-L5 were cleaned of soft tissue, and decorticated with a high-speed burr (See FIG. 1). The crushed autograft was then spread over and between the transverse processes at the appropriate level (L4-L5). An equivalent amount of implant, or blank was incorporated into the autograft bed (See FIG. 2). Retractors were removed and the paraspinal muscles were allowed to cover the fusion bed. The dorsal lumbar fascia was closed using a running 4-0 resorbable suture and the skin was closed with interrupted 4-0 resorbable sutures. The surgical site was treated with antibiotic ointment, and the rats were given a dose of Enrofloxacin antibiotic (10 mg/kg). Radiographs were taken immediately after surgery. Blood glucose levels were taken before surgery, and 12 and 24 hours after surgery. See Table 1.

TABLE 1
Systemic Blood Glucose Levels (mg/dL)

| | Before | 12 | 24 |
|---|---|---|---|
| Group | surgery | hours | hours |
| Controls | | | 91.4 |
| VAC-low | 103.5 | 213.4 | 117.7 |
| VAC-high | 102.9 | 153.2 | 90.7 |
| Zn-low | 106.0 | 122.8 | 101.8 |
| Zn-high | 109.3 | 120.0 | 89.0 |

### Pellet Preparation

In order to prepare the pellets, 0.2 mL of each stock solution was mixed with 0.4 g of CaSO₄ to obtain the appropriate consistency of the carrier in a 1 mL syringe. It was then be injected into 2 mm diameter clear Tygon laboratory tubing and allowed to harden overnight.

Once set, pellets were sectioned into 7 mm pieces and autoclaved (to sterilize), prior to implantation.

Assumption: Weight of SD rat = 0.45 kg

| | Vn (0.75 | Vn (1.5 | Zn (0.5 | Zn (1.0 |
|---|---|---|---|---|
| | mg/kg) | mg/kg) | mg/kg) | mg/kg) |
| Mass of | 0.338 mg | 0.675 mg | 0.225 mg | 0.45 mg |
| treatment for | | | | |
| each rat | | | | |

In order to prepare the stock solution, the volume of solution in each pellet was calculated by using the volume ratio of solution to mixture.

### Volume of CaSO₄ in Each Mixture

D CaSO₄=2.96 g/cm³

$$( 0.4 {g\_CaSO}_{4} ) / ( 2.96 \frac{g}{{cm}^{3}} ) = 0.135 {cm}^{3} = 0.135 mL$$

### Volume of Mixture and Ratio

0.135 mL CaSO₄+0.2 mL solution=0.335 mL mixture

0.2 mL solution/0.335 mL mixture×100%=59.7% solution per mixture

### Volume of Each Pellet, 1 mm Radius, 7 mm Height

V=πr²h,=π(1 mm)²(7 mm)=22 mm³=0.022 mL

### Volume of Solution in Each Pellet

0.022 mL×59.7%=0.0131 ml solution per pellet

### Stock Solution (10 mL)

Because bilateral surgery is performed, mass of treatment (X) must be halved for each pellet.

$$( ( \frac{X}{2} ) / 0.0131 mL ) \times 10$$

| | Vn (0.75 | Vn (1.5 | Zn (0.5 | Zn (1.0 |
|---|---|---|---|---|
| | mg/kg) | mg/kg) | mg/kg) | mg/kg) |
| Mass of | 129.0 mg | 258.0 mg | 85.9 mg | 171.8 mg |
| treatment in | | | | |
| each stock | | | | |
| solution (10 ml) | | | | |

### Radiographic Analysis

Posteroanterior radiographs at 35 kV for 90 seconds were taken at 8 weeks after sacrifice and harvest. All soft tissue was removed prior to radiographic exam. Two blinded independent observers graded the radiographs as solid fusion mass bilaterally (A), unilateral fusion mass (B), small fusion mass bilaterally (C), and graft resorption (D), based on previously published radiographic scales.

### Manual Palpation

After removal of all soft tissue, two blinded independent observers manually palpated and stressed across the fusion site (L4-L5). Specimens were graded as fused (A), partially fused (B), and not fused (C).

### Quantitative MicroCT Analysis

Spines harvested at 8 weeks also underwent a micro-CT analysis to quantitatively calculate new bone formation. Areas of interest were demarcated from the top of the L4 transverse process cephalad to the bottom of the L5 transverse process caudally, including any bone lateral to a vertical line connecting the pairs of the involved vertebrae. The cubic millimeters of bone in these areas of interest (bilaterally) for each specimen were quantified using micro-CT. A Skyscan 1172 High Resolution MicroCT (Skyscan, Kontich, Belgium) was used with a pixel size of 17.4 micrometers.

### Statistical Analysis

A two-sample t test was performed to determine the significance of blood glucose levels, and bone volume on microCT. A Mann-Whitney Rank Test was performed for analysis of radiographs and manual palpation. Kappa values were calculated for inter-rater agreement. Statistical analysis was performed using SigmaStat.

### Results

Of the 50 animals, one of the control rats died on postoperative day one, likely due to anaesthesia. The remaining 49 rats had no complications and were sacrificed as planned (0.02% perioperative mortality rate).

### Radiographic Analysis

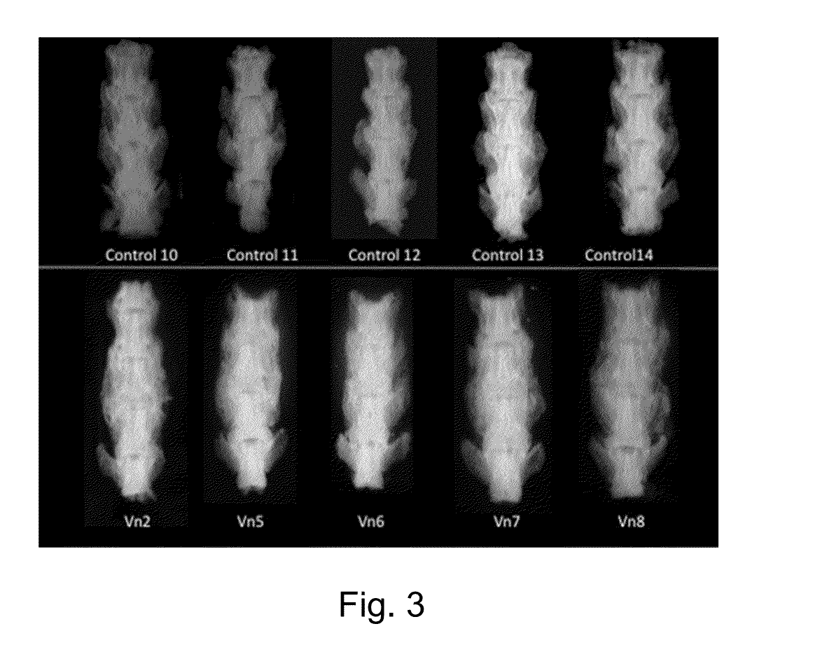

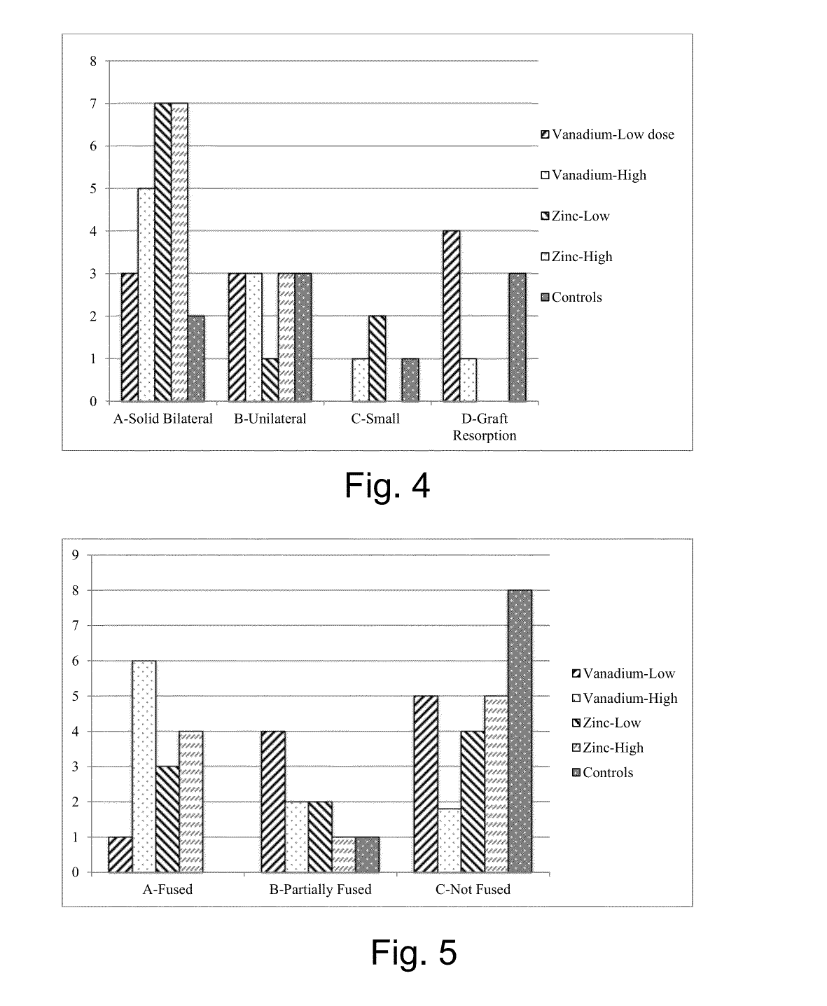

Based on radiographs, examples of which are shown in FIG. 3, in the high dose vanadium group 5/10 had solid fusion mass bilaterally, 3/10 had unilateral fusion, 1/10 had small fusion mass bilaterally, and 1/10 had graft resorption (p=0.270, kappa=0.667). The low dose vanadium group had 3/10 solid fusion mass bilaterally, 3/10 had unilateral fusion, 0/10 had small fusion mass bilaterally, and 4/10 had graft resorption (p=0.807, kappa=0.583). The high dose zinc group had 7/10 solid fusion mass bilaterally, 3/10 had unilateral fusion, 0/10 had small fusion mass bilaterally, and 0/10 had graft resorption (p=0.05, kappa=1.0). The low dose zinc group had 7/10 solid fusion mass bilaterally, 1/10 had unilateral fusion, 2/10 had small fusion mass bilaterally, and 0/10 had graft resorption (p=0.066, kappa=0.512). The control group had 2/9 solid fusion mass bilaterally, 3/9 unilateral fusion, 1 small fusion mass bilaterally, and 3/9 had graft resorption(kappa=0.297). See Table 2 and FIG. 4.

TABLE 2
Radiographs

| Group | A | B | C | D | Kappa | P Value |
|---|---|---|---|---|---|---|
| Controls | 2 | 3 | 1 | 3 | 0.297 | |
| VAC—low | 3 | 3 | 0 | 4 | 0.583 | 0.807 |
| VAC—high | 5 | 3 | 1 | 1 | 0.667 | 0.270 |
| Zn—low | 7 | 1 | 2 | 0 | 0.512 | 0.066 |
| Zn—high | 7 | 3 | 0 | 0 | 1.0 | 0.050 |

A = solid fusion mass bilaterally
B = unilateral fusion mass
C = small fusion mass bilaterally
D = Graft resorption

### Manual Palpation Test

Based on manual palpation, in the high dose Vanadium group 6/10 had solid fusion, 2/10 were partially fused, and 2/10 were not fused (p=0.002, kappa=0.412). In the low dose vanadium group, 1/10 had solid fusion, 4/10 were partially fused, and 5/10 were not fused (p=0.072, kappa=0.130). In the high dose Zinc group, 4/10 had solid fusion, 1/10 had partially fused, and 5/10 were not fused (p=0.008, kappa=0.306). In the low dose Zinc group, 3/10 had solid fusion, 4/10 had partially fused, and 3/10 were not fused (p=0.055, kappa=0.565). In the control group, 0/9 had solid fusion, 1/9 had partially fused, and 8/9 were not fused (kappa=0.156). See Table 3 and FIG. 5.

TABLE 3
Manual Palpation

| Group | A | B | C | Kappa | P Value |
|---|---|---|---|---|---|
| Controls | 0 | 1 | 8 | 0.156 | |
| VAC-low | 1 | 4 | 5 | 0.130 | 0.072 |
| VAC-high | 6 | 2 | 2 | 0.412 | 0.002 |
| Zn-low | 3 | 4 | 3 | 0.565 | 0.055 |
| Zn-high | 4 | 1 | 5 | 0.306 | 0.008 |

A = fused
B = partially fused
C = not fused

### Quantitative Micro-CT Analysis

Based on MicroCT analysis, the mean bone volume of the L4/L5 transverse processes and fusion mass for controls was 126.7 mm³. The high dose Vanadium group had mean 170.8 mm³, and in the low dose Vanadium group had mean 167.4 mm³. The high dose Zinc group had a mean of 172.7 mm³, and the low dose Zinc group had a mean of 172.9 mm^{A} (see table 4).

TABLE 4
Mean Bone Volume (mm3) n MicroCT

| | Mean Bone | Std | |
|---|---|---|---|
| Group | Volume mm³ | Dev | P value |
| Table 4a | | | ANOVA p = 0.006 |
| Vn high dose | 170.8 | 37.1 | <0.01 vs control |
| (n = 10) | | | |
| Vn low dose | 167.4 | 23.5 | <0.05 vs control |
| (n = 10) | | | |
| Controls | 126.7 | 26.3 | |
| (n = 9) | | | |
| Table 4b | | | ANOVA p = 0.002 |
| Zn high dose | 172.7 | 26.4 | <0.01 vs control |
| (n = 10) | | | |
| Zn low dose | 172.9 | 31.6 | <0.01 vs control |
| (n = 10) | | | |
| Controls | 126.7 | 26.3 | |
| (n = 9) | | | |

### Summary of Results

Compared with controls, the high dose zinc group demonstrated a significantly higher manual palpation grade (p=0.008), radiographic score (p=0.05), and bone formation on microCT (172.7 mm³ vs. 126.7 mm³ for controls) (p<0.01). The low dose zinc trended towards significantly higher manual palpation (p=0.055), and radiographic scores (p=0.066) and had significantly more bone formed on microCT (172.9 mm) (p<0.01) compared with controls. The high dose vanadium had significantly higher manual palpation scores (p=0.002) and bone formation on MicroCT (170.8 mm³) (p<0.01), and no difference in radiographic scores (p=0.270). Low dose vanadium had significantly more bone on microCT (172.9 mm³) (p<0.05), trended towards higher scores on manual palpation (p=0.072) and had no difference on radiographic scores (p=0.807).

### Discussion of Results

Pseudarthrosis following spinal fusion procedures is an undesirable outcome, and local adjuncts to help prevent this complication are of significant interest. This study demonstrates the potential benefit of a local insulin-mimetic agent applied to the fusion bed in a rat posterolateral intertransverse lumbar fusion model. To our knowledge, this is the first study to examine the effects of local zinc or vanadium to lumbar spinal fusion in a rat model.

Several studies have demonstrated the insulin-like effects of vanadium and zinc, including the effects of oral administration. The majority of these animal studies have demonstrated some benefit of oral vanadium on bone quality in diabetic animals, however, not all studies are in agreement. The results of the various studies may demonstrate some benefit to oral administration of vanadium compounds, mostly in diabetic rats. The focus of our investigation, however, was to examine the local effects of vanadium on bone formation in spinal fusion in non-diabetic rats.

The effects of vanadium on fracture healing and cartilage formation have also been studied. The mechanism by which vanadium exerts its insulin-like properties is believed to include activation of key components of the insulin signaling pathway, in addition to enhancing insulin sensitivity and prolonging insulin action. (Vardatsikos, G., et al. (2009). “Bis(Maltolato)-Oxovanadium (IV)-Induced Phosphorylation Of PKB, GSK-3 And FOXOI Contributes To Its Glucoregulatory Responses (review).” Int J Mol Med 24(3): 303-309). While our study did not investigate the mechanism by which vanadium influences spinal fusion, it may exert similar effects as insulin, which has also been demonstrated to improve fracture healing and spinal fusion in rat models.

The potential toxic effects of vanadium are concerning, and have been studied. Local administration could avoid some of the concerns of toxicity to other organs. Local administration to a fracture or fusion site could decrease accumulation in other tissues seen after oral administration, however to our knowledge, this has not been studied.

Zinc has been recognized to be insulin-mimetic in the form of zinc chloride in its ability to stimulate lipogenesis in rat adipocytes, (Coulston, L. and P. Dandona (1980). “Insulin-Like Effect Of Zinc On Adipocytes.” Diabetes 29(8): 665-667) and numerous studies have been done since demonstrating its relation to diabetes. Vardatsikos et al recently performed an in depth review of the insulin-mimetic and anti-diabetic effects of zinc. (Vardatsikos, G., et al. (2012). “Insulino-Mimetic And Anti-Diabetic Effects Of Zinc.” J Inorg Biochem 120C: 8-17). The mechanism by which zinc exerts insulin-like effects is believe to include activation of insulin signaling pathways including extracellular signal-related kinase 1/2, and phosphatidylinositol 3-kinase/protein kinase B/Akt pathways. (Vardatsikos et al. 2012). These may be similar mechanisms to which zinc enhances spinal fusion in a rat model, however our study did not investigate this.

A limitation of this study is that the mechanism by which zinc and vanadium effect spinal fusion was not examined. Also, the interobserver reliability for radiographic and manual palpation scoring was lower in the control group compared to each of the test groups. The low interobserver reliability may be due to difficulty in scoring specimens that were either “partially fused” or “not fused”. A scoring system with only two grades, “fused” or “not fused” may have provided higher interobserver reliability. While we tested two different insulin-mimetic agents at two dosages, we are unable to make definitive conclusions for which group was superior. At the time of harvest, some of the pellets had not completely dissolved, and some were partially incorporated into the fusion site. The clinical effects of this observation are unknown, and future studies will determine the optimal carrier and dosage.

Based on this study, both zinc and vanadium demonstrated better fusion rates compared to controls at both dosages tested. While the fusion rate of our control group was low, this is comparable to other autograft fusion rates in a rat model. (Dimar, J. R., 2nd, et al. (1996). “The Effects Of Nonsteroidal Anti-Inflammatory Drugs On Posterior Spinal Fusions In The Rat.” Spine (Phila Pa 1976) 21(16): 1870-1876); (Wang, J. C., et al. (2003). “Effect Of Regional Gene Therapy With Bone Morphogenetic Protein-2-Producing Bone Marrow Cells On Spinal Fusion In Rats.” J Bone Joint Surg Am 85-A(5): 905-911); (Grauer, J. N., et al. (2004). “Posterolateral Lumbar Fusions In Athymic Rats: Characterization Of A Model.” The Spine Journal: Official Journal Of The North American Spine Society 4(3): 281-286) and (Drespe, I. H., et al. (2005). “Animal Models For Spinal Fusion.” The Spine Journal: Official Journal Of The North American Spine Society 5(6 Suppl): 209S-216S). Radiographically, both zinc groups had significantly higher fusion rates, and interobserver reliability was high. Manual palpation is often considered the gold standard to determine fusion in small animal models, and the high dose vanadium group performed best in this test. In an effort to eliminate some of the subjective nature of radiographic and manual palpation scoring, MicroCT was used to quantitatively determine new bone formation. Each test group scored significantly higher than the control group.

This study is the first to examine the effects of local insulin-mimetics in a rat spinal fusion model. The results are promising, and future work will focus on the optimal dosage and carrier, as well as examining the mechanism by which insulin-mimetics affect spinal fusion.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

All references cited hereby are incorporated by reference in their entirety.

1. A bone tissue material for facilitating fusion of vertebrae in a spinal fusion surgical procedure, the material comprising an insulin-mimetic agent selected from the group consisting of insulin mimetic zinc, vanadium, tungsten, molybdenum, niobium, selenium and manganese compounds and combinations thereof.

2-3. (canceled)

4. The bone tissue material according to claim 1, the material further comprising a pharmaceutically acceptable carrier.

5. The bone tissue material of claim 4, wherein said pharmaceutically acceptable carrier is an inorganic salt.

6. The bone tissue material of claim 4, wherein said pharmaceutically acceptable carrier is an inorganic salt selected from sulfates and phosphates.

7. The bone tissue material of claim 4, wherein said pharmaceutically acceptable carrier is a calcium salt.

8. A method of enhancing spinal fusion in a spinal fusion surgical procedure, the method comprising the steps of:
exposing a portion of each of adjacent vertebrae; and
placing supplementary bone tissue material and an insulin-mimetic agent within an area between the exposed portions of the adjacent vertebrae and in contact with the exposed portions of both vertebrae;
wherein the insulin-mimetic agent is selected from the group consisting of insulin-mimetic zinc, vanadium, tungsten, molybdenum, niobium, selenium and manganese compounds and combinations thereof, and is provided in an amount effective to increase the rate of fusion of the two vertebrae with the bone tissue material.

9-10. (canceled)

11. The method according to claim 8, wherein the insulin-mimetic agent is added directly to the fusion site.

12. The method according to claim 8, wherein the insulin-mimetic agent is added in a form of bone tissue material formulated to be suitable for implantation.

13. The method according to claim 8, wherein the insulin-mimetic agent is added as a composition further comprising a surgically acceptable carrier.

14. The method of claim 13, wherein the composition is an insulin-mimetic calcium sulfate pellet.

15. The method according to claim 8, wherein said placing step further comprises placing an allograft or a ceramic bone-graft substitute with said supplementary bone tissue material and an insulin-mimetic agent within said area between the exposed portions of the adjacent vertebrae and in contact with said exposed portions of both vertebrae.

16. The method according to claim 8, wherein said placing step comprises implanting an interbody device with said supplementary bone tissue material and said insulin-mimetic agent within said area between the exposed portions of the adjacent vertebrae and in contact with said exposed portions of both vertebrae.

17. A bone tissue kit for facilitating fusion of vertebrae in a spinal fusion surgical procedure, comprising a composition formulated for facile application in a spinal fusion procedure comprising an insulin-mimetic agent selected from the group consisting of zinc, vanadium, tungsten, molybdenum, niobium, selenium and manganese compounds and combinations thereof and a pharmaceutically acceptable carrier.

18. The bone tissue kit of claim 17, further comprising allograft bone tissue material and/or ceramic bone-graft substitute.

19. The bone tissue kit of claim 18, wherein the insulin-mimetic agent and the allograft bone tissue material or ceramic bone-graft substitute are provided in a mixture.

20. The bone tissue kit of claim 18, wherein the insulin-mimetic agent and allograft bone tissue material or ceramic bone-graft substitute are provided for subsequent mixing.

21-23. (canceled)

24. An implantable device for enhancing spinal fusion, comprising a prosthetic implant configured to stabilize and promote the fusion of two adjacent vertebrae, wherein the bone tissue contacting surfaces of the prosthetic implant are coated with a composition comprising an insulin-mimetic agent selected from the group consisting of zinc, vanadium, tungsten, molybdenum, niobium, selenium and manganese compounds and combinations thereof.

25. The device of claim 24, wherein said prosthetic implant is configured to supply autograft bone, allograft bone or ceramic bone-graft substitute to exposed surfaces of the two adjacent vertebrae.

26. (canceled)

27. The device of claim 24, wherein said composition further comprises a calcium phosphate, a calcium sulfate, or combinations thereof.

28. The device of claim 24, wherein said composition further comprises a ceramic bone graft substitute.
